(19) United States
(12) Patent Application Publication
(10) Pub. No.: US 2025/0295364 A1
(43) Pub. Date: Sep. 25, 2025

(54) SYSTEM AND METHOD FOR ASSESSING THE QUALITY OF ECG DATA

(71) Applicant: eResearch Technology, Inc., Philadelphia, PA (US)

(72) Inventors: Hui Zhao, Philadelphia, PA (US); Robert Pastor, Philadelphia, PA (US); Jean-Philippe Couderc, Philadelphia, PA (US); Luc Dekie, Philadelphia, PA (US); Vickas Patel, Philadelphia, PA (US)

(73) Assignee: eResearch Technology, Inc., Philadelphia, PA (US)

(21) Appl. No.: 18/899,852

(22) Filed: Sep. 27, 2024

Related U.S. Application Data
(60) Provisional application No. 63/568,271, filed on Mar. 21, 2024.

(51) Int. Cl. A61B 5/00; A61B 5/318; A61B 5/339

(52) U.S. Cl. CPC A61B 5/7221; A61B 5/318; A61B 5/339; A61B 5/7475

(57) ABSTRACT

Exemplary system and methods for assessing ECG data quality. The system includes a processor that decomposes a continuous ECG signal into multiple epochs and generates a quality score for each epoch. A first rolling window of a first specified width is applied to the decomposed ECG signal to capture plural quality scores from a sequence of epochs in the plural epochs. The processor computes a local metric from the plural quality scores captured by the first rolling window. The processor captures plural quality scores and computes associated local metrics across an entirety of the decomposed ECG signal. Once completed the processor executes one or more application modules for: generating a quality alert signal and generating a quality visualization signal for displaying a color map corresponding to signals associated with a local metric. The output of the one or more executed application modules is passed to a user interface.

### RELATED APPLICATIONS

This application claims priority to U.S. Application No. 63/568,271 filed on Mar. 21, 2024, the entire content of which is hereby incorporated by reference.

### FIELD

The subject matter disclosed relates generally to artificial intelligence and machine learning, and particularly to a model trained for assessing the quality of an electrocardiogram signal.

### BACKGROUND

In clinical medicine, an electrocardiogram (ECG) provides more information about patient safety than any other single assessment. Assuring the ECG data are usable and complete in drug development allows for reliable assessment of the effect of a new compound on the QT interval, other ECG parameters and morphological changes. As discussed in ICH E14. “Questions & Answers (R3),” December 10, 2015, https://database.ich.org/sites/default/files/E14_Q % 26As_R3_Q%26As.pdf, concentration-response analysis, in which all available data across all doses are used to characterize the potential for a drug to influence correct QT interval (QTc), can serve as an alternative to the by-time-point analysis or intersection-union test as the primary basis for decisions to classify the risk of a drug. Concentration-response data would not necessarily come from a dedicated QT study. Data can be acquired from first-in human studies, multiple-ascending dose studies, or other studies but requires robust, high-quality ECG recording and analysis sufficient to support a valid assay for ECG intervals. If the strategy selected is to collect, clean and store the ECG data for later analysis (e.g., once the pharmacokinetics of the drug have been assessed and the drug candidate passes proof of concept), then quality checking the full dataset at the time of ECG data collection can improve the likelihood the future analysis will provide meaningful results. See Darpo et al. ECG Evaluation as Part of the Clinical Pharmacology Strategy in the Development of New Drugs: A Review of Current Practices and Opportunities Based on Five Case Studies. The Journal of Clinical Pharmacology 2022;62(12): 1480-500. https://doi.org/10.1002/jcph.2095.

Reviewing and assessing the quality of extensive continuous ECG data can be a laborious task. Nonetheless, both the current availability of powerful computerized tools, such as artificial intelligence (Al) and machine learning (ML), and the existence of extensive ECG repositories have made this challenge more attainable. In addition, the standardized signal-based nature of the ECG lends itself well to Al/ML modeling and automation for handling large datasets. The marriage of Al/ML and ECGs has ushered in a new era of cardiac healthcare, promising precision, efficiency and the potential for improving trial outcomes. AI/ML applications are gaining increasing relevance, especially with the growing adoption of wearable devices such as ECG patches and other ambulatory devices, making it an area of considerable importance for cardiac monitoring.

In more recent years, AI/ML has also become a natural extension of the classical signal processing paradigm, in which the linear processing blocks are replaced by non-linear equivalents that enable scientists to manage a much broader set of problems. See e.g., Askin et al. “Artificial Intelligence Applied to Clinical Trials: Opportunities and Challenges.” Health and Technology 2023;13(2): 203-13. https://doi.org/10.1007/s12553-023-00738-2. One area of interest is the ability to quickly respond to the collection of ECG tracings and signals in case the quality is unacceptable or to provide a benchmark of ECG quality for large sets of data.

These algorithms are typically based on deep learning (DL) network architectures comprising multiple hidden layers, as discussed by Mathew et al. “Deep Learning Techniques: An Overview.” In Advanced Machine Learning Technologies and Applications, edited by Hassanien A E, Bhatnagar R, Darwish A, 1141:599-608. Singapore: Springer Singapore, 2021. The most frequently used DL algorithms are convolutional neural networks (CNNs), which were originally proposed for object recognition and image classification. However, AI/ML tools can also be useful to rapidly assess data quality and support signal processing tasks.

WO 2013/036718 discloses a technique in which signal quality can be measured using Kurtosis or spectral density analysis techniques. The amount of useful information provided in a signal is directly proportional to the degree of Gaussian distribution in the data. The analysis can determine an amount of deviation from a template, such as measuring the cross-correlation of a signal metric against an average signal metric template. Here, a low correlation between the data and a template indicates low quality data.

CN 115005835 discloses a technique for assessing the quality of a signal in which an ECG signal is filtered to obtain a frequency band of 3 to 45 Hz. A quality index is calculated on the filtered signal. The quality index is divided by the index threshold to obtain an index threshold classification result. The index threshold classification result is used to fuse and evaluate the ECG signal data so that a quality assessment of the ECG signal data can be made. The technique distinguishes clean ECG signal data from contaminated data and classifies the ECG data as being excellent, good, or failing.

CN 112971801 discloses a system and method for evaluating the quality of a continuous physiological signal. The system includes a signal segmentation unit that receives an original continuous physiological signal and divides the signal into segments using a windowing technique. A preprocessing unit filters the signal using at least one of known bandpass or median filtering techniques. A feature extraction unit extracts feature values from the filtered signals. An evaluation unit receives the extracted feature values and generates a score that is evaluated based on predetermined thresholds. The signal is determined to be of good, medium, or poor quality based on the evaluation result.

CN 114176519 discloses a method for determining the quality of a non-contact ECG signal. The non-contact ECG signal is acquired by a device and is processed to clean and segment the signal into 5 second intervals. The processing further includes labeling and classifying the signal as a clear signal that can be used clinically, an ambiguous signal that needs further processing to extract attributes of the ECG waveform, or a clinically unusable signal characterized by baseline drift and large noise. Features are extracted from the classified signal and used to generate a feature matrix. The feature matrix is used to generate a training dataset and a test dataset for an AI/ML model.

CN 108090509 discloses a system and method in which an ECG signal is segmented, where each segment is formatted to a standard time length. The ECG segment data is identified and classified to determine whether the data is good or abnormal. An accuracy rate assessment is performed to assess the validity of the classification.

### SUMMARY

An exemplary method for assessing quality of an electrocardiogram (ECG) signal is disclosed, the method comprising: a) decomposing, by a processor, a continuous ECG signal into plural epochs, wherein each epoch is of a specified length; b) generating, by the processor, a quality score for each epoch generated from the decomposed ECG signal; c) applying, by the processor, a first rolling window of a first specified width to the decomposed ECG signal to capture plural quality scores from a sequence of epochs in the plural epochs; d) computing, by the processor, a local metric from the plural quality scores captured by the first rolling window; repeating steps c) and d) across an entirety of the decomposed ECG signal; executing, by the processor, one or more application modules configured for: generating a quality alert signal including streaming ECG data obtained from at least one portion of the received ECG signal and a notification based on the local metric associated with the at least one portion of the received ECG signal; generating a quality visualization signal for displaying a color map for one or more segments of the received ECG signal, the color maps corresponding to a local metric associated with the one or more segments of the received ECG signal; and sending, by the processor, an output of the one or more executed application modules to a user interface.

An exemplary system for assessing quality of an electrocardiogram (ECG) signal is disclosed, the system comprising: a processor encoded with program code, which when executed causes the processor to be configured to perform the operations of: a trained epoch model configured to decompose a continuous ECG signal into plural epochs, and generate a quality score for each epoch; a metric generation module configured to: iteratively apply a first rolling window of a first specified width to the decomposed ECG signal to capture plural quality scores from a sequence of epochs in the plural epochs and compute a local metric from the plural quality scores captured by the first rolling window; and one or more application modules configured to: generate a quality alert signal including streaming ECG data obtained from at least one portion of the received ECG signal and a notification based on the local metric associated with the at least one portion of the received ECG signal; generate a quality visualization signal for displaying a color map for one or more segments of the received ECG signal, the color maps corresponding to a local metric associated with the one or more segments of the received ECG signal; and the processor further configured to send an output of the one or more executed application modules to a user interface.

A non-transitory computer readable medium encoded with program code for assessing quality of an electrocardiogram (ECG) signal, the computer readable medium when brought into communicable contact with a processor, causes the processor to be configured to perform the operations of: a) decomposing a continuous ECG signal into plural epochs, wherein each epoch is of a specified length; b) generating a quality score for each epoch generated from the decomposed ECG signal; c) applying a first rolling window of a first specified width to the decomposed ECG signal to capture plural quality scores from a sequence of epochs in the plural epochs; d) computing a local metric from the plural quality scores captured by the first rolling window; repeating steps c) and d) across an entirety of the decomposed ECG signal; executing one or more application modules configured for: generating a quality alert signal including streaming ECG data obtained from at least one portion of the received ECG signal and a notification based on the local metric associated with the at least one portion of the received ECG signal; generating a quality visualization signal for displaying a color map for one or more segments of the received ECG signal, the color maps corresponding to a local metric associated with the one or more segments of the received ECG signal; and sending an output of the one or more executed application modules to a user interface.

### DESCRIPTION OF THE DRAWINGS

Exemplary embodiments are best understood from the following detailed description when read in conjunction with the accompanying drawings. Included in the drawings are the following figures:

FIG. 1A illustrates a system for assessing quality of an electrocardiogram (ECG) signal in accordance with an exemplary embodiment of the present disclosure.

FIGS. 1B to 1D illustrate a deep learning neural network in accordance with an exemplary embodiment of the present disclosure.

FIG. 2A illustrates an epoch in accordance with an exemplary embodiment of the present disclosure.

FIG. 2B illustrates the flow of input data used to generate the output data in accordance with an exemplary embodiment of the present disclosure.

FIGS. 3A and 3B illustrate a sequence of epochs and epoch quality scores generated in accordance with an exemplary embodiment of the present disclosure.

FIG. 4 illustrates a sequence of local metrics generated in accordance with an exemplary embodiment of the present disclosure.

FIG. 5 illustrates a graph of occurrence metrics generated in accordance with an exemplary embodiment of the present disclosure.

FIG. 6 illustrates a color map generated in accordance with an exemplary embodiment of the present disclosure.

FIG. 7 illustrates a method for assessing quality of an electrocardiogram (ECG) signal in accordance with an exemplary embodiment of the present disclosure.

FIG. 8 illustrates hardware/software components of a computing device configured for assessing quality of an electrocardiogram (ECG) signal in accordance with an exemplary embodiment of the present disclosure.

Further areas of applicability of the present disclosure will become apparent from the detailed description provided hereinafter. It should be understood that the detailed descriptions of exemplary embodiments are intended for illustration purposes only and, therefore, are not intended to necessarily limit the scope of the disclosure.

### DETAILED DESCRIPTION

In accordance with exemplary embodiments of the present disclosure, systems and methods one or more machine learning (ML) or artificial intelligence (AI) models are trained to perform an algorithm that assesses the quality of continuous electrocardiogram (ECG) recordings. The exemplary systems and methods can be implemented to assess ECG recordings during one or more phases or studies of a clinical trial. The technology described herein decomposes a continuous ECG signal into plural epochs and captures plural quality scores from a sequence of epochs. The quality scores are measured by calculating a local metric and the data is processed to generate a quality alert signal based on at least one portion of the ECG signal and to generate a color map of one or more segments of the ECG signal. The outputs can be used to identify issues in quality within the ECG signal and/or identify inconsistencies or errors in data used in one or more phases or studies of a clinical trial, which allow for corrective action(s) to be taken to address the errors.

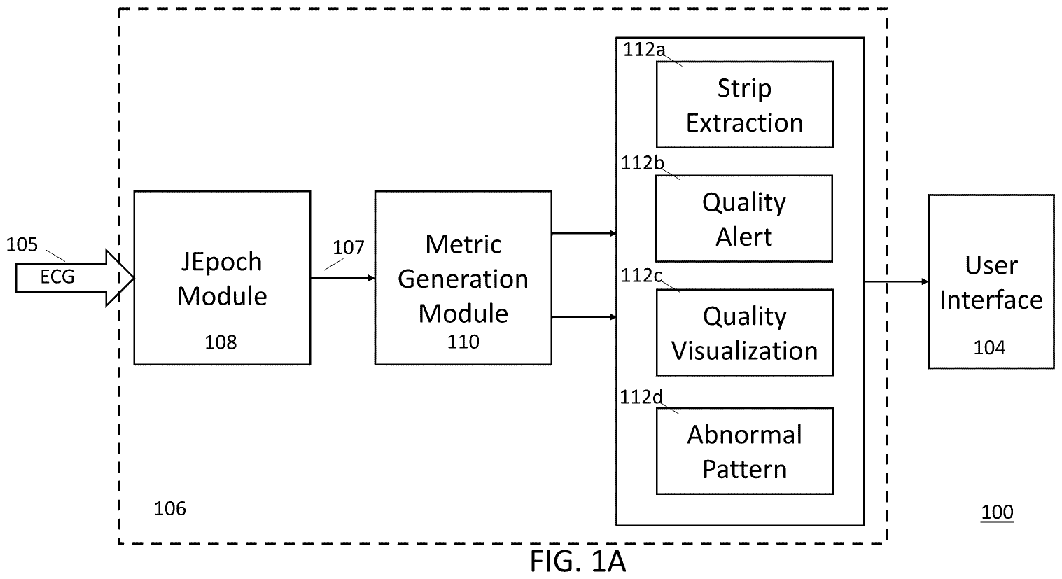

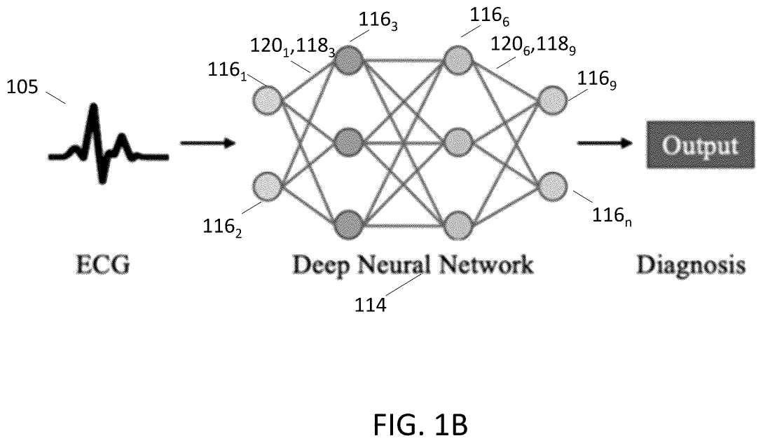

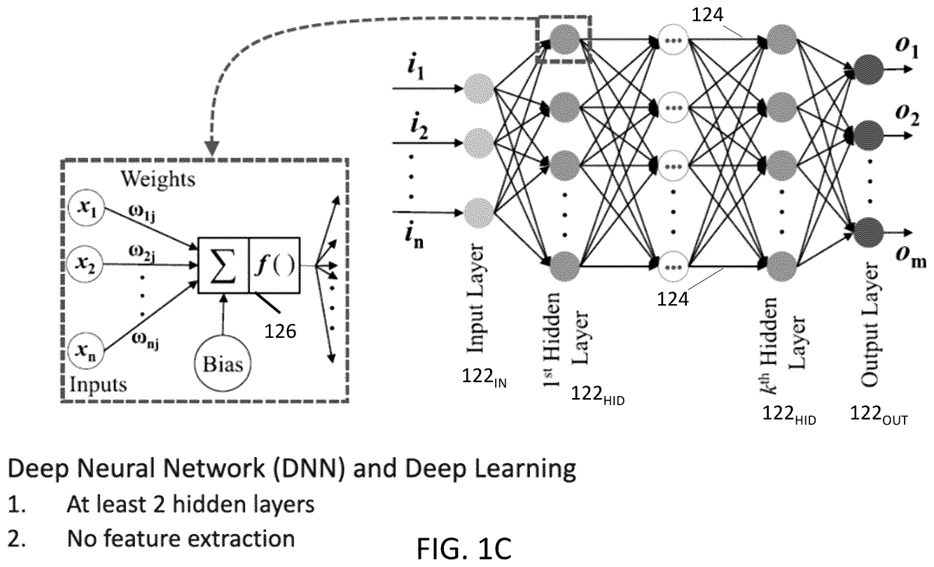

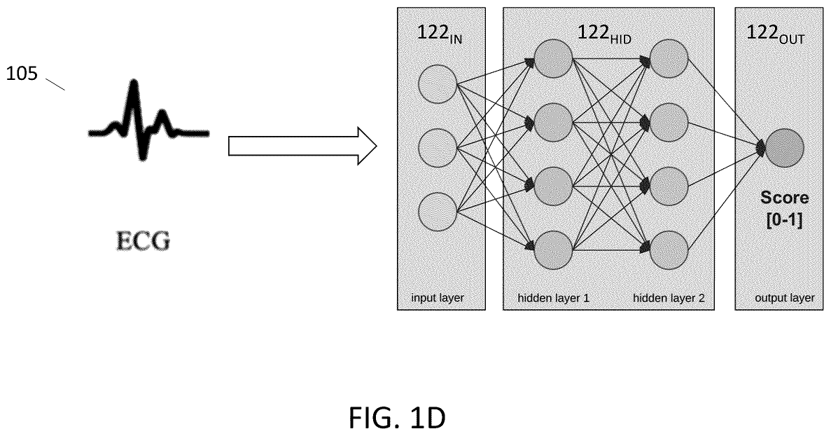

FIG. 1A illustrates a system for assessing quality of an electrocardiogram (ECG) signal in accordance with an exemplary embodiment of the present disclosure.

As shown in FIG. 1A, the system 100 can be configured as a computing system having a user interface 104. The computing system 100 can include a processor 106 configured to execute program code for assessing quality of an electrocardiogram (ECG) signal 105. Executing the program code, causes the processor 106 to be configured with an epoch module 108 and a metric generation module 110, and one or more application modules 112a to 112d for generating an output based on the measured quality of the ECG signal.

The epoch module 108 and metric generation module 110 can include one or more artificial intelligence (AI) or machine learning models (ML) 114. FIGS. 1B to 1D illustrate a deep learning neural network in accordance with an exemplary embodiment of the present disclosure. The algorithms for analyzing the ECG signal are based on deep learning (DL) network architectures, such as convolutional neural networks (CNNs). Neural networks can include plural nodes that represent individual computational units. Each node has one or more biased input/output connections that function as transfer or activation functions for combining the inputs and outputs in a specified manner. As shown in FIG. 1B the neural network 114 includes plural nodes 116₁ to 116ₙ where each node 116ₙ has one or more inputs 118 and outputs 120 for processing the input ECG signal 105. The neural network 114 is formed by an arrangement of the plural nodes 116ₙ into multiple layers 122, the scheme within which the nodes 116, are connected determines the type and operation of the neural network 114. For example, as shown in FIG. 1C, the neural network 114 can include an input layer 122_{IN}, multiple hidden layers 122_{HID}, and an output layer 122_{OUT}. Each layer 122 may perform a different or specified transformation on the respective inputs, using a different or specified mathematical calculation or function. Signals travel or are passed between the layers 122, from the input layer 122_{IN} to the output layer 122_{OUT} via the middle or hidden layers 122_{HID} and can traverse any layer 122 and node(s) 114 multiple times. As shown in FIG. 1C, the nodes 116n can be connected in an array and each node can transmit a signal to a node in another layer 122 of the neural network 114. The input/output connections 118, 120 between the nodes have a corresponding weight Wₙⱼ and are combined according to the bias applied at each node. For example, the connections 124 are activation or transfer functions which trigger the respective nodes and combine inputs according to mathematical equations or formulas 126 according to the bias. According to these neural network principles, and as shown in FIG. 1D, the ECG signal 105 is received at an input layer 122_{IN} of the neural network 114 and passed through multiple hidden layers 122_{HID} until an epoch score 304 and/or local metric 404 is generated at the output layer 122_{OUT}. No feature extraction is performed on the signal as it is passed through the multiple layers/nodes of the neural network 114.

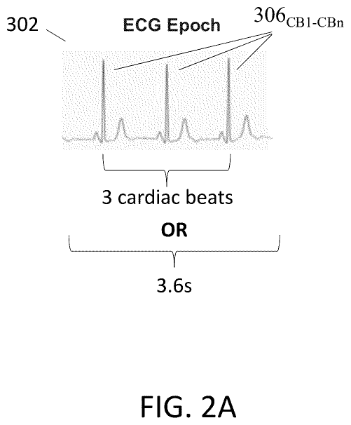

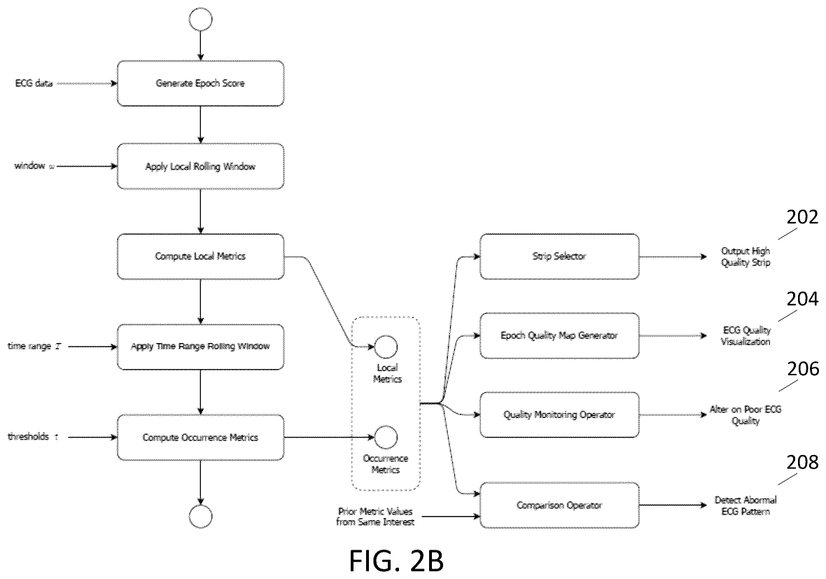

FIG. 2A illustrates an epoch in accordance with an exemplary embodiment of the present disclosure. FIG. 2B illustrates a signal processing flow in accordance with an exemplary embodiment of the present disclosure. The epoch module 108 can be configured to receive the continuous ECG signal 105 as an input and decomposes the continuous ECG signal 105 into plural epochs 302 and generates a quality score 304 for each epoch 302. According to exemplary embodiments described herein, the epoch module can be configured to generate each epoch according to a specified epoch window length. The selected window length provides a balance between a desired time resolution and data context for determining quality. The window length specifies the smallest unit of measure for the ECG signal 105 can be captured. An epoch window of a short duration, e.g., 3 seconds or less, allows for an increased scaling of an epoch based on time. However, the increased time resolution limits the ability of the window to also provide a broader context of the captured data relative to a broader portion of the ECG signal 105. According to exemplary embodiments of the present disclosure, the epoch window can be set to capture 1 cardiac beat or set to a length of 1.2 seconds. According to another exemplary embodiment, the epoch window can be set the capture a least 3 cardiac beats or set to a duration of 3.6 seconds. For example, according to an exemplary embodiment, each epoch includes plural cardiac beats 306_{CB1-CB}_{ₙ} and is centered around one of the plural cardiac beats 306_{CB2}. The epoch represents an interval that is centered around a specific beat during which signal stability and clarity have a potential to yield accurate measurements. According to yet another exemplary embodiment, the epoch module can be configured such that multiple epoch windows of different lengths or durations can be used to capture data. For example, the epoch module can be configured such that multiple epoch windows are applied to the ECG signal 105 according to a specified sequence or at specified intervals until an entirety of the ECG signal 105 has been processed.

The trained AI-model can be configured to extract ECGs from a continuous recording (e.g., a Holter recording) acquired from a Holter monitor, such as a portable or wearable device. For example, protocol-specific extraction windows can be used to extract up to 10-non-overlappying digital 12-lead ECG tracings (e.g., 14 second) from the continuous Holter recording. The extracted ECG tracings are input to the trained AI-model, which assesses the quality of the ECG tracings based on the epoch quality scores and metrics computed based on the epoch quality scores. The ECGs can be obtained using various types of lead systems including, a 1-lead ECG system, a 3-lead ECG system, a 12-lead ECG system, a vector ECG, and EASI lead system, a 360° lead system, or any other suitable system for generating an ECG as desired.

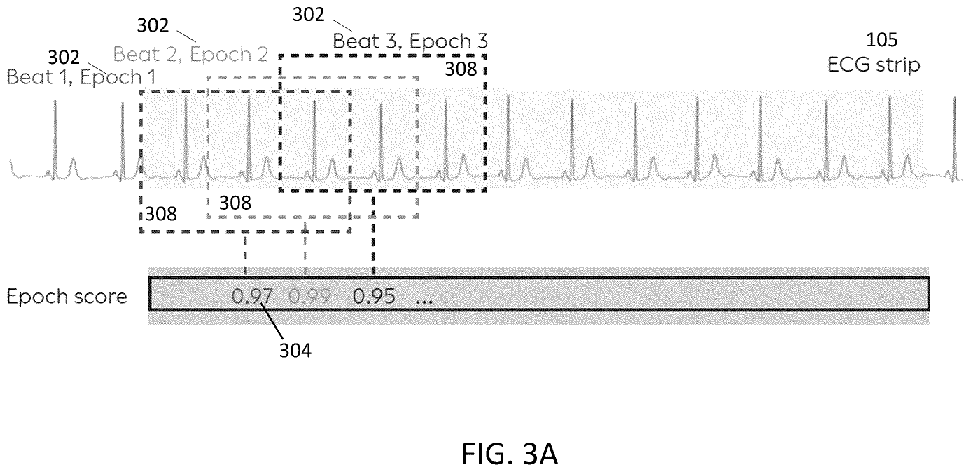

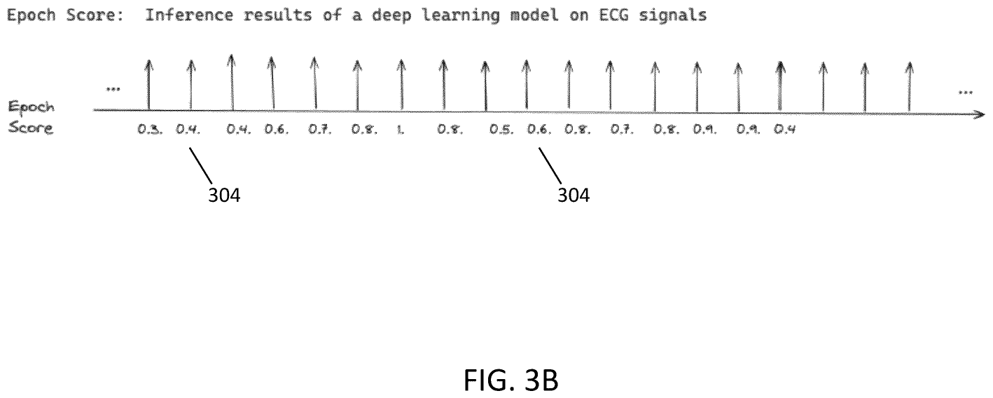

FIGS. 3A and 3B illustrate a sequence of epochs 302 and epoch quality scores 304 generated in accordance with an exemplary embodiment of the present disclosure. According to an exemplary embodiment, the quality score 304 for each epoch 302 has a value range from 0 to 1, where 1 represents a higher or best quality score and 0 represents a lower or poor quality score. The epoch window length defines the smallest duration of the ECG signal 105 for which the quality score is computed. As shown in FIG. 3A, the epoch window 308 can be specified to capture three beats for calculating the quality or epoch score 304.

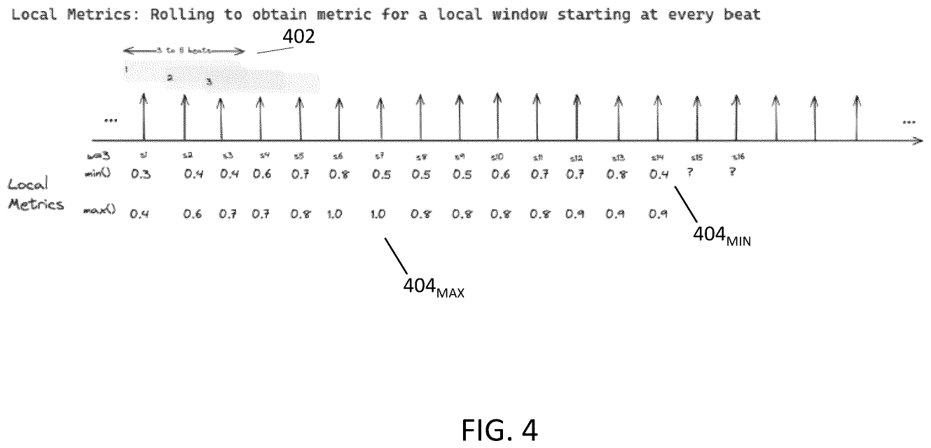

FIG. 4 illustrates a sequence of local metrics generated in accordance with an exemplary embodiment of the present disclosure. As shown in FIG. 4, a local or first rolling window 402 is applied to each epoch window 308 of FIG. 3A and local metrics 404 are computed based on the data within each window 308. For example, the metric generation module 110 can be configured to iteratively apply the first rolling window 402 of a first specified width to the decomposed ECG signal to capture plural quality scores 304 from a sequence of epochs 302, which are defined by window 308, and compute a local metric 404 from the plural quality scores 304 captured by the first rolling window 402. According to an exemplary embodiment, the first rolling window 402 can be iteratively applied over an entirety of the decomposed ECG signal 107. According to another exemplary embodiment, the first rolling window 402 can be iteratively applied over a portion of the decomposed ECG signal 107 according to a specified time period. The period can be defined by a range having a specified start time and stop time determined by a user. The quality scores capture and define a local pattern for the portion of ECG signal 107 within the first rolling window 402. The length of the first rolling window 402 defines the coverage of the local metric 404. Furthermore, the first rolling window 404 is applied to all epoch scores 304 within the local window duration to compute the local metrics 404 starting at each beat.

As shown in FIG. 4, the metric generation module 110 can be configured to determine a minimum epoch quality score 404_{MIN} for each epoch 302 within the first rolling window 402. According to another exemplary embodiment, the metric generation module 110 is configured to determine a maximum epoch score 404_{MAX} for each epoch 302 within the first rolling window 402. In yet another exemplary embodiment, the metric generation module 110 is configured to determine a standard deviation of epoch quality scores among plural epochs 302 within the first rolling window 402. According to an exemplary embodiment, the metric generation module 110 can be configured to determine an average epoch score for the plural epochs 302 within the first rolling window 402. It should be understood that the metric generation module can be configured to generate one or more of the minimum epoch quality score 304_{MIN}, the maximum epoch score 304_{MAX}, the average epoch score, and the standard deviation of epoch quality scores using the same first rolling window 402. According to another exemplary embodiment, the metric generation module 110 can be configured to perform separate iterative applications of the first rolling window 402 to the decomposed ECG signal 107 for generating each of the minimum epoch quality score 304_{MIN}, the maximum epoch score 304_{MAX}, the average epoch score, and the standard deviation of epoch quality scores.

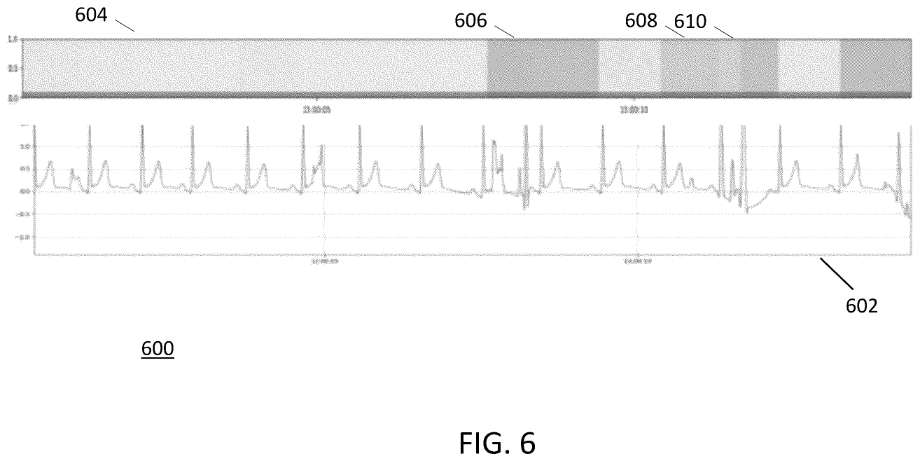

As shown in FIGS. 1 and 2, the processor 106 can be further configured to execute one or more application modules 112a-112d for generating an output that visualizes a result of the ECG quality determination based on the local metric calculation. For example, according to an exemplary embodiment the processor 106 can be configured to generate a quality alert signal including streaming ECG data obtained from at least one portion of the received ECG signal 105 and a notification based on the local metric associated with the at least one portion of the received ECG signal. The streaming ECG data can be a strip of data that includes a specified portion of the ECG data defined by a time range and/or defined by the result of the local metric calculations. FIG. 6 illustrates a color map generated in accordance with an exemplary embodiment of the present disclosure. As shown in FIG. 6, the color or gradient map 600 is graph that shows plural time points and an epoch quality score distribution for a specified ECG strip 602. According to an exemplary embodiment, the ECG strip 602 can be identified and/or designated by a user based on a defined time range and/or score distribution. For example, the color map can be associated with an ECG strip having the highest average epoch score or highest the standard deviation of epoch quality scores. As shown in FIG. 6, the various color or shaded areas of the color map can be associated with a quality level of epoch quality scores within the ECG strip 602. For example, shaded area 604 represents a low noise ECG signal, shaded area 606 represents an ECG signal with artifacts, shaded area 608 represents an ECG signal with lesser artifacts that shaded area 606, and shaded area 610 represents an ECG signal having a period of intense artifact and noise activity, where the duration of shaded area 610 is much less than the duration of an ECG signal identified in other portions of the ECG strip 602. According to yet another exemplary embodiment, computing device can use the one or more applications to generate an abnormal pattern signal comparing a current local metric with a previous local metric obtained from memory.

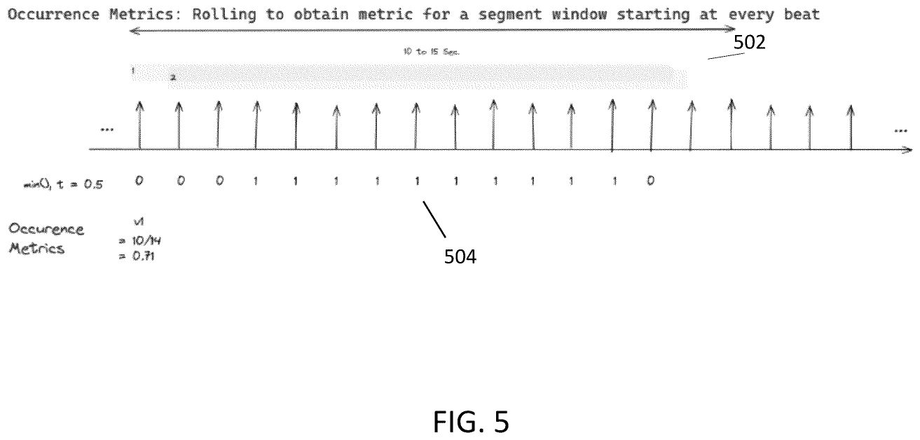

As shown in FIG. 5, the metric generation module 110 can be configured to apply a time range or second rolling window 502 of a second specified width to the decomposed ECG signal 107 to capture plural occurrence metrics 504 associated with a sequence of epochs in the plural epochs 302. The occurrence metric 504 is used to describe a segment of the ECG signal 105 determined by the length or duration of the second rolling window 502. For example, the metric generation module is configured to generate the occurrence metrics 504 by computing a local descriptor value at each beat. The metric generation module can apply specified thresholds to any of the local metrics 404 to generate a local descriptor for each applied threshold. Based on the local descriptor value, the metric generation module generates the occurrence metric 504 for the ECG segment captured in the second rolling window 502. FIG. 5 illustrates a graph of occurrence metrics 504 generated in accordance with an exemplary embodiment of the present disclosure. As shown in FIG. 5, the second rolling window 502 with length of a specified strip duration and is applied iteratively at every beat of the decomposed ECG signal 107. For example, the second rolling window 502 can have a duration of at least the width of the first rolling window. According to another exemplary embodiment, the length of the second rolling window can range from 10 seconds to 15 seconds, or any other suitable length or range as desired. The occurrence metric 504 measures the occurrence of each local metric value generated in FIG. 4, within the second rolling window 502 by computing a local descriptor value (i.e. 1) for every beat that is greater than or equal to 0.5. According to an exemplary embodiment and as shown in FIG. 5, the second rolling window 502 can compute a local descriptor for a sequence of local metrics, such as, a percentage computation related to the number of local metrics in the second rolling window 502 which exhibit the minimum epoch quality score 304 MIN or the maximum epoch score 304_{MAX}.

Once the occurrence metric 504 has been generated, the computing device can, through the one or more applications already discussed, extract a signal strip of fixed duration from the received ECG signal. For one or more ECG signal segments, the metric generation module can be configured to compute a highest mean occurrence metric 504 among plural occurrence metrics. According to another exemplary embodiment, the computing device can execute a comparison operator that identifies an abnormal pattern signal by comparing a current occurrence metric with a previous occurrence metric stored in memory.

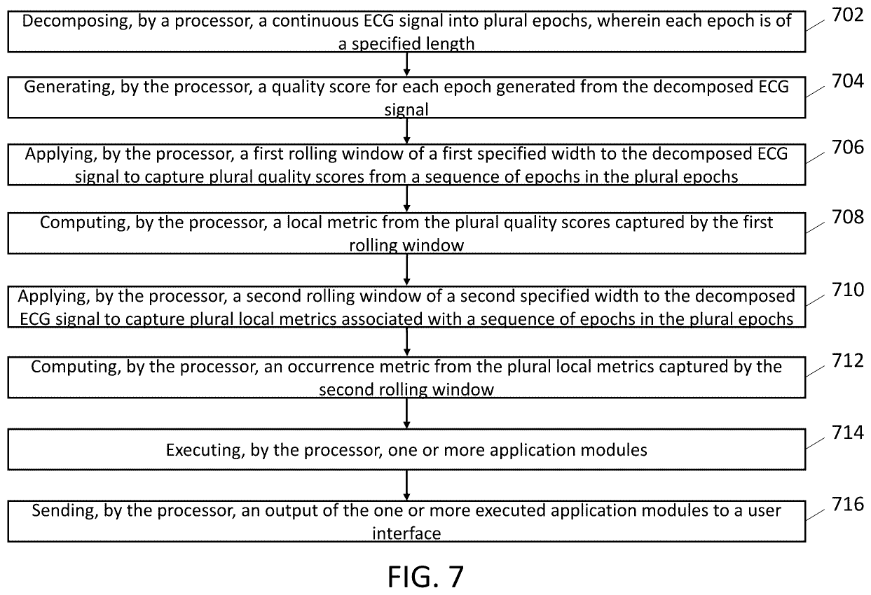

FIG. 7 illustrates a method for assessing quality of an electrocardiogram (ECG) signal in accordance with an exemplary embodiment of the present disclosure.

As shown in FIG. 7, the processor 106 of the computing device 100 can execute programming code for performing the method for assessing the quality of an electrocardiogram (ECG) signal. Upon executing the programming code, the processor 106 of the computing device 100 can perform the step of decomposing a continuous ECG signal into plural epochs (S702). According to an exemplary embodiment, the processor 106 can be configured to define the epoch based on the number of beats, e.g., 1 or more, or a specified length of time, e.g., 1.2 seconds, 3.6 seconds, or other suitable duration as desired. As already discussed, the properties of the epoch are selected to balance resolution and context of the quality determination and for signal stability and clarity. In step S704, the processor 106 generates a quality score for each epoch generated from the decomposed ECG signal 107. The processor 106 applies a first rolling window 402 of a first specified width to the decomposed ECG signal 107 to capture plural quality scores from a sequence of epochs in the plural epochs (Step S706). In step S708, the processor 106 computes a local metric 404 from the plural quality scores captured by the first rolling window 402. The processor 106 applies a second rolling window 502 of a second specified width to the decomposed ECG signal 107 to capture plural local metrics 404 associated with a sequence of epochs in the plural epochs (Step S710). In step S712, the processor 106 computes an occurrence metric 504 from the plural local metrics 404 captured by the second rolling window 502. The first rolling window 402 for generating the local metric 404 and the second rolling window 502 for generating the occurrence metric 504 are applied iteratively until the entirety of the decomposed ECG signal 107 has been processed. After the entire decomposed

ECG signal 107 has been processed, the processor 106 executes one or more application modules 112a-112d (Step S714). For example, the one or more application modules 112aa-112d can be used for generating a quality alert signal 206 including streaming ECG data obtained from at least one portion of the received ECG signal 105 and a notification based on the local metric 404 associated with the at least one portion of the received ECG signal, generating a quality visualization signal 204 for displaying a color map for one or more segments 107 of the received ECG signal 105, the color maps corresponding to a local metric 404 associated with the one or more segments 107 of the received ECG signal 105, extracting a signal strip 202 of fixed duration from the received ECG signal 105, the signal strip having a highest mean occurrence metric among plural occurrence metrics of the decomposed ECG signal 107, and generating an abnormal pattern signal 208 comparing at least one of a current local metric and current occurrence metric with a previous local metric and a previous occurrence metric, respectively. In step S716, the processor sends an output of the one or more executed application modules to a user interface. For example, one of the application modules can include an application programming interface that allows the computing device to communicate with a corresponding API being executed on a user device using a specified set of known definitions and protocols.

The exemplary system and methods of the present disclosure can be implemented using a number and arrangement of systems, hardware, and/or modules (e.g., software instructions). For example, the system can be a combination of two or more systems, hardware, and/or modules or may be implemented within a single system, hardware, and/or module. A single system, hardware, and/or module may be implemented as multiple, distributed systems, hardware, and/or modules. Additionally, or alternatively, a set of systems, a set of hardware, and/or a set of modules (e.g., one or more systems, one or more hardware devices, one or more modules) may perform one or more functions described as being performed by another set of systems, another set of hardware, or another set of modules.

The system can be implemented in a configuration suitable for analyzing the quality of an ECG signal as disclosed herein. For example, various components of the system may be implemented in one or more computing devices (e.g., one or more servers, client devices, user devices, and/or the like) and the one or more computing devices may be connected via a communications network (e.g., the Internet).

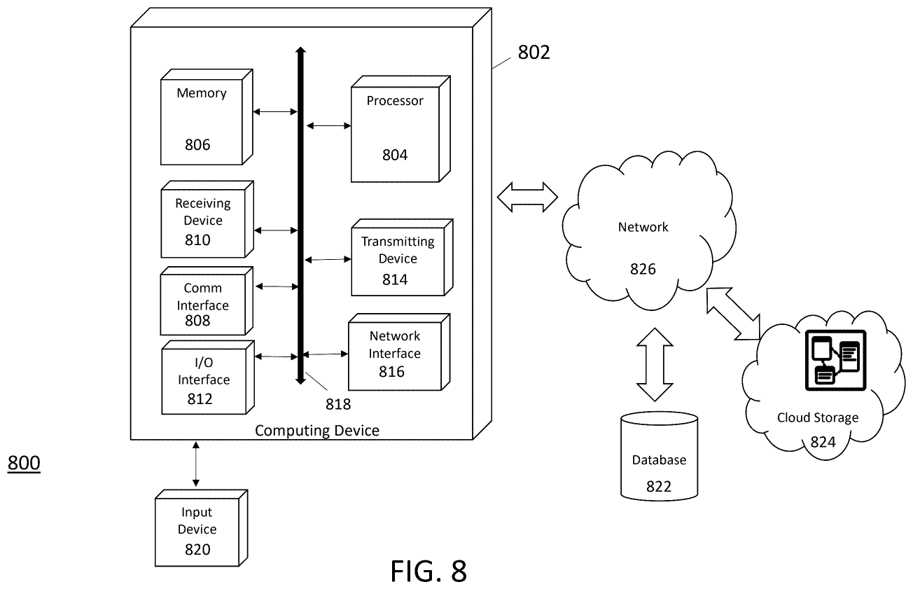

FIG. 8 illustrates hardware/software components of a computing device configured for assessing quality of an electrocardiogram (ECG) signal in accordance with an exemplary embodiment of the present disclosure. An exemplary system 800 as disclosed herein, can be configured for training machine learning and/or artificial intelligence models (e.g., neural models, neural networks, and/or the like) and for determining the quality of an ECG signal with trained machine learning models. The system 800 may include computing device 802. The computing device 802 may include a processor 804 (e.g., CPU) and memory 806. The processor 804 may execute software instructions (e.g., program code) for determining the quality of an ECG signal.

The processor 804 may be implemented in hardware, software, or a combination of hardware and software. For example, the processor 804 may include a common processor (e.g., a CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed and/or execute software instructions to perform a function.

Memory 806 may include random access memory (RAM), read-only memory (ROM), and/or another type of dynamic or static storage device (e.g., flash memory, magnetic memory, optical memory, etc.) that stores information and/or software instructions for use by the processor. Memory 806 may include a computer-readable medium and/or storage component. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 806 from another computer-readable medium or from another device via a communication interface with a computing device. When executed, software instructions stored in memory may cause the processor 804 to perform one or more processes described herein. Embodiments described herein are not limited to any specific combination of hardware circuitry and software.

Any of the processors disclosed herein can include any integrated circuit or other electronic device (or collection of devices) capable of performing an operation on at least one instruction, which can include a Reduced Instruction Set Core (RISC) processor, a CISC microprocessor, a Microcontroller Unit (MCU), a CISC-based Central Processing Unit (CPU), a Digital Signal Processor (DSP), a Graphics Processing Unit (GPU), a Field Programmable Gate Array (FPGA), etc. The hardware of such devices may be integrated onto a single substrate (e.g., silicon “die”), or distributed among two or more substrates. Various functional aspects of the processor may be implemented solely as software or firmware associated with the processor.

The processor 804 can include one or more processing or operating modules. A processing or operating module can be a software or firmware operating module configured to implement any of the functions disclosed herein. The processing or operating module can be embodied as software and stored in memory. The memory can be operatively associated with the processor. A processing module can be embodied as a web application, a desktop application, a console application, etc.

The processor 804 can include or be associated with a computer or machine readable medium. The computer or machine readable medium can include memory. Any of the memory discussed herein can be computer readable memory configured to store data. The memory 806 can include a volatile or non-volatile, transitory or non-transitory memory, and be embodied as a database 822, an active memory, a cloud memory, cloud storage, or cloud servers 824, or any other suitable device or system as desired. Examples of memory 806 can include flash memory, Random Access Memory (RAM), Read Only Memory (ROM), Programmable Read only Memory (PROM), Erasable Programmable Read only Memory (EPROM), Electronically Erasable Programmable Read only Memory (EEPROM), FLASH-EPROM, Compact Disc (CD)-ROM, Digital Optical Disc DVD), optical storage, optical medium, a carrier wave, magnetic cassettes, magnetic tape, magnetic disk storage or other magnetic storage devices, or any other medium which can be used to store the desired information and which can accessed by the processor 804.

The memory 806 can be a non-transitory computer-readable medium. The term “computer-readable medium” (or “machine-readable medium”) as used herein is an extensible term that refers to any medium or any memory, which participates in providing instructions to the processor for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, transmission media, etc. The computer or machine readable medium can be configured to store one or more instructions thereon. The instructions can be in the form of algorithms, program logic, etc. that cause the processor to execute any of the functions disclosed herein.

Embodiments of the memory 806 can include a processor module and other circuitry to allow for the transfer of data to and from the memory, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc. Communications can be via Bluetooth, near field communications, cellular communications, telemetry communications, Internet communications, etc.

Data stored in the exemplary computing device 802 (e.g., in the memory) can be stored on any type of suitable computer readable media, such as optical storage (e.g., a compact disc, digital versatile disc, Blu-ray disc, etc.), magnetic tape storage (e.g., a hard disk drive), or solid-state drive. An operating system can also be stored in the memory 806.

In an exemplary embodiment, the data can be configured in any type of suitable database configuration, such as a relational database, a structured query language (SQL) database, a distributed database, an object database, etc. Suitable configurations and storage types will be apparent to persons having skill in the relevant art.

The exemplary computing device 802 can also include a communications interface 808. The communications interface 808 can be configured to allow software and data to be transferred between the computing device and external devices. Exemplary communications interfaces can include a modem, a network interface (e.g., an Ethernet card), a communications port, a PCMCIA slot and card, etc. Software and data transferred via the communications interface 808 can be in the form of signals, which can be electronic, electromagnetic, optical, or other signals as will be apparent to persons having skill in the relevant art. The signals can travel via a communications path, which can be configured to carry the signals and can be implemented using wire, cable, fiber optics, a phone line, a cellular phone link, a radio frequency link, etc. Transmission of data and signals can be via transmission media. Transmission media can include coaxial cables, copper wire, fiber optics, etc. Transmission media can also take the form of acoustic or light waves, such as those generated during radio-wave and infrared data communications, or other forms of propagated signals (e.g., carrier waves, digital signals, etc.).

Memory semiconductors (e.g., DRAMs, etc.) can be means for providing software to the computing device. Computer programs (e.g., computer control logic) can be stored in the memory. Computer programs can also be received via the communications interface. Such computer programs, when executed, can enable computing device to implement the present methods as discussed herein. In particular, the computer programs stored on a non-transitory computer-readable medium, when executed, can enable hardware processor devices to implement the methods as discussed herein. Accordingly, such computer programs can represent controllers of the computing device.

An exemplary computing device or system for performing the operations disclosed herein may include at least one computing device and/or at least one component of computing device.

The computing system or device may further include a receiver or receiving device, a network interface, an input/output (I/O) interface 812, a transmitting device 814, a communication infrastructure 818, and an input device 820.

The receiver or receiving device 810 may be a combination of hardware and software components configured to receive data samples from the mobile network or database. According to exemplary embodiments, the receiving device 810 can include a hardware component such as an antenna, a network interface (e.g., an Ethernet card), a communications port, a Personal Computer Memory Card International Association (PCMCIA) slot and card, 5G New Radio (NR) interface, or any other component or device suitable for use on a mobile communication network or Radio Access Network as desired. The receiving device 810 can be an input device for receiving signals and/or data samples formatted according to 3GPP protocols and/or standards. The receiving device 810 can be connected to other devices via a wired or wireless network 826 or via a wired or wireless direct link or peer-to-peer connection without an intermediate device or access point. The hardware and software components of the receiving device can be configured to receive the data from the mobile network according to one or more communication protocols and data formats. For example, the receiving device 810 can be configured to communicate over a network, which may include a local area network (LAN), a wide area network (WAN), a wireless network (e.g., Wi-Fi), a mobile communication network, a satellite network, the Internet, fiber optic cable, coaxial cable, infrared, radio frequency (RF), another suitable communication medium as desired, or any combination thereof. During a receive operation, the receiving device can be configured to identify parts of the received data via a header and parse the data signal and/or data packet into small frames (e.g., bytes, words) or segments for further processing at the processor.

The processor 804 can be configured for executing the program code stored in memory 806. Upon execution, the program code causes the processor to perform the functions at a node on the mobile communication network or remote computing device (e.g., server, computer, etc.) of the user and executes program code to assessing the quality of ECG data on the mobile communication network according to the exemplary embodiments described herein. The processor can be a special purpose, or a general purpose computing device encoded with program code or software for performing the exemplary functions and/or features disclosed herein. According to exemplary embodiments of the present disclosure, the processor can include a CPU. The CPU can be connected to the communications infrastructure including a bus, message queue, or network, multi-core message-passing scheme, for communicating with other components of the computing system, such as the memory, input device, the communications interface 808, and the I/O interface 812. The CPU can include one or more processors such as a microprocessor, microcomputer, programmable logic unit or any other suitable hardware computing devices as desired.

According to exemplary embodiments described herein, the combination of the memory 806 and the processor 804 can store and/or execute computer program code for performing the specialized functions described herein. The program code can be stored on a non-transitory computer readable medium, such as the memory devices for the computing device, which may be memory semiconductors (e.g., DRAMs, etc.) or other tangible and non-transitory means for providing software to the computing device. For example, via any known or suitable service or platform, the program code can be deployed (e.g., streamed and/or downloaded) remotely from computing devices located on a local-area or wide-area network and/or in a cloud-computing arrangement or environment. In another example, the computer programs (e.g., computer control logic) or software may be stored in memory resident on/in the computing device. The computer programs or software may be stored in a computer program product or non-transitory computer readable medium and loaded into the computing device using any one or combination of a removable storage drive, an interface for internal or external communication, and a hard disk drive, where applicable. The computer programs or software, when executed, may enable the computing device to implement the present methods and exemplary embodiments discussed herein. Accordingly, such computer programs may represent controllers of the computing device.

The I/O interface 812 can be configured to receive the signal from the processor and generate an output suitable for a peripheral device via a direct wired or wireless link. The I/O interface 812 can include a combination of hardware and software for example, a processor, circuit card, or any other suitable hardware device encoded with program code, software, and/or firmware for communicating with a peripheral device such as a display device, printer, audio output device, or other suitable electronic device or output type as desired.

The transmitting device 814 can be configured to receive data from the processor and assemble the data into a data signal and/or data packets according to the specified communication protocol and data format of a peripheral device or remote device to which the data is to be sent. The transmitting device 814 can include any one or more of hardware and software components for generating and communicating the data signal over the communications infrastructure and/or via a direct wired or wireless link to a peripheral or remote device. The transmitting device 814 can be configured to transmit information according to one or more communication protocols and data formats as discussed in connection with the receiving device 810.

According to exemplary embodiments described herein, the memory 806 and the processor 804 can store and/or execute computer program code for performing the specialized functions described herein. It should be understood that the program code can be stored on a non-transitory computer readable medium, such as the memory devices for the system (e.g., computing device), which may be memory semiconductors (e.g., DRAMs, etc.) or other tangible non-transitory means for providing software to the system. The computer programs (e.g., computer control logic) or software may be stored in memory devices (e.g., device memory) resident on/in the system. The computer programs may also be received from external storage devices and/or network storage locations via a communications interface. Such computer programs, when executed, may enable the system to implement the present methods and exemplary embodiments discussed herein. Accordingly, such computer programs may represent controllers of the system. Where the present disclosure is implemented using software, the software may be stored in a computer program product or non-transitory computer readable medium and loaded into the system using any one or combination of a removable storage drive, an interface for internal or external communication, and a hard disk drive, where applicable.

In the context of exemplary embodiments of the present disclosure, a processor 802 can include one or more modules or engines configured to perform the functions of the exemplary embodiments described herein. Each of the modules or engines may be implemented using hardware and, in some instances, may also utilize software, such as corresponding to program code and/or programs stored in memory. In such instances, program code may be interpreted or compiled by the respective processors (e.g., by a compiling module or engine) prior to execution. For example, the program code may be source code written in a programming language that is translated into a lower level language, such as assembly language or machine code, for execution by the one or more processors and/or any additional hardware components. The process of compiling may include the use of lexical analysis, preprocessing, parsing, semantic analysis, syntax-directed translation, code generation, code optimization, and any other techniques that may be suitable for translation of program code into a lower level language suitable for controlling the system to perform the functions disclosed herein. It will be apparent to persons having skill in the relevant art that such processes result in the system being a specially configured computing device uniquely programmed to perform the functions of the exemplary embodiments described herein.

It will be appreciated by those skilled in the art that the present invention can be embodied in other specific forms without departing from the spirit or essential characteristics thereof. The presently disclosed embodiments are therefore considered in all respects to be illustrative and not restrictive. The scope of the invention is indicated by the appended claims rather than the foregoing description and all changes that come within the meaning and range and equivalence thereof are intended to be embraced therein.

What is claimed is:

1. A method for assessing quality of an electrocardiogram (ECG) signal, the method comprising:
a) decomposing, by a processor, a continuous ECG signal into plural epochs, wherein each epoch is of a specified length;
b) generating, by the processor, a quality score for each epoch generated from the decomposed ECG signal;
c) applying, by the processor, a first rolling window of a first specified width to the decomposed ECG signal to capture plural quality scores from a sequence of epochs in the plural epochs;
d) computing, by the processor, a local metric from the plural quality scores captured by the first rolling window;
repeating steps c) and d) across an entirety of the decomposed ECG signal;
executing, by the processor, one or more application modules configured for:
generating a quality alert signal including streaming ECG data obtained from at least one portion of the received ECG signal and a notification based on the local metric associated with the at least one portion of the received ECG signal;
generating a quality visualization signal for displaying a color map for one or more segments of the received ECG signal, the color maps corresponding to a local metric associated with the one or more segments of the received ECG signal; and
sending, by the processor, an output of the one or more executed application modules to a user interface.

2. The method of claim 1, wherein each epoch includes plural cardiac beats and is centered around one of the plural cardiac beats.

3. The method of claim 2, wherein each epoch includes at least 1 cardiac beat and/or has a duration of at least 1.2 seconds.

4. The method of claim 1, wherein the quality score for each epoch has a value range from 0 to 1.

5. The method of claim 1, wherein computing the local metric comprises determining a minimum epoch score within the first rolling window.

6. The method of claim 1, wherein computing the local metric comprises determining a maximum epoch score within the first rolling window.

7. The method of claim 1, wherein computing the local metric comprises determining an average epoch score within the first rolling window.

8. The method of claim 1, wherein computing the local metric comprises determining a standard deviation of epoch scores within the first rolling window.

9. The method of claim 1, further comprising:
e) applying, by the processor, a second rolling window of a second specified width to the decomposed ECG signal to capture plural local metrics associated with a sequence of epochs in the plural epochs; and
f) computing, by the processor, an occurrence metric from the plural local metrics captured by the second rolling window; and
repeating steps e) and f) across an entirety of the decomposed ECG signal.

10. The method of claim 9, wherein the second rolling window has a duration of at least a width of the first rolling window.

11. The method of claim 9, wherein the occurrence metric has a value range from 0 to 1.

12. The method of claim 9, further comprising:
executing, by the processor, one or more application modules configured for:
extracting a signal strip of fixed duration from the received ECG signal, the signal strip having a highest mean occurrence metric among plural occurrence metrics of the decomposed ECG signal.

13. The method of claim 9, further comprising:
executing, by the processor, one or more application modules configured for:
generating an abnormal pattern signal comparing at least one of a current local metric and current occurrence metric with a previous local metric and a previous occurrence metric, respectively.

14. A system for assessing quality of an electrocardiogram (ECG) signal, the system comprising:
a processor encoded with program code, which when executed causes the processor to be configured to perform the operations of:
a trained epoch model configured to decompose a continuous ECG signal into plural epochs, and generate a quality score for each epoch;
a metric generation module configured to:
iteratively apply a first rolling window of a first specified width to the decomposed ECG signal to capture plural quality scores from a sequence of epochs in the plural epochs and compute a local metric from the plural quality scores captured by the first rolling window; and
one or more application modules configured to:
generate a quality alert signal including streaming ECG data obtained from at least one portion of the received ECG signal and a notification based on the local metric associated with the at least one portion of the received ECG signal;
generate a quality visualization signal for displaying a color map for one or more segments of the received ECG signal, the color maps corresponding to a local metric associated with the one or more segments of the received ECG signal; and
the processor further configured to send an output of the one or more executed application modules to a user interface.

15. The system of claim of claim 14, wherein each epoch includes plural cardiac beats and is centered around one of the plural cardiac beats.

16. The system of claim 15, wherein each epoch includes at least 1 cardiac beat and/or has a duration of at least 1.2 seconds.

17. The system of claim 15, wherein the quality score for each epoch has a value range from 0 to 1.

18. The system of claim 15, wherein to compute the local metric, the metric module is configured to determine a minimum epoch score within the first rolling window.

19. The system of claim 15, wherein to compute the local metric, the metric module is configured to determine a maximum epoch score within the first rolling window.

20. The system of claim 15, wherein to compute the local metric, the metric module is configured to determine an average epoch score within the first rolling window.

21. The system of claim 15, wherein to compute the local metric, the metric module is further configured to determine a standard deviation of epoch scores within the first rolling window.

22. The system of claim 15, wherein the second rolling window has a duration of at least a width of the first rolling window.

23. The system of claim 14, wherein the one or more applications modules is further configured to:
iteratively apply a second rolling window of a second specified width to the decomposed ECG signal to capture plural local metrics associated with a sequence of epochs in the plural epochs and compute an occurrence metric from the plural local metrics captured by the second rolling window.

24. The system of claim 23, further comprising one or more application modules configured to:
extract a signal strip of fixed duration from the received ECG signal, the signal strip having a highest mean occurrence metric among plural occurrence metrics of the decomposed ECG signal; and
generate an abnormal pattern signal comparing at least one of a current local metric and current occurrence metric with a previous local metric and a previous occurrence metric, respectively.

25. A non-transitory computer readable encoded with program code for assessing quality of an electrocardiogram (ECG) signal, the computer readable medium when brought into communicable contact with a processor, causes the processor to be configured to perform the operations of:
a) decomposing a continuous ECG signal into plural epochs, wherein each epoch is of a specified length;
b) generating a quality score for each epoch generated from the decomposed ECG signal;
c) applying a first rolling window of a first specified width to the decomposed ECG signal to capture plural quality scores from a sequence of epochs in the plural epochs;
d) computing a local metric from the plural quality scores captured by the first rolling window;
repeating steps c) and d) across an entirety of the decomposed ECG signal;
executing one or more application modules configured for:
generating a quality alert signal including streaming ECG data obtained from at least one portion of the received ECG signal and a notification based on the local metric associated with the at least one portion of the received ECG signal;
generating a quality visualization signal for displaying a color map for one or more segments of the received ECG signal, the color maps corresponding to a local metric associated with the one or more segments of the received ECG signal; and
sending an output of the one or more executed application modules to a user interface.

26. The non-transitory computer readable medium of claim 25, wherein the processor is further configured to perform the operations of:
e) applying a second rolling window of a second specified width to the decomposed ECG signal to capture plural local metrics associated with a sequence of epochs in the plural epochs; and
f) computing an occurrence metric from the plural local metrics captured by the second rolling window;
repeating steps e) and f) over an entirety of the decomposed ECG signal.

27. The non-transitory computer readable medium of claim 25, wherein the processor is further configured to perform the operations of:
executing one or more application modules configured for:
extracting a signal strip of fixed duration from the received ECG signal, the signal strip having a highest mean occurrence metric among plural occurrence metrics of the decomposed ECG signal; and
generating an abnormal pattern signal comparing at least one of a current local metric and current occurrence metric with a previous local metric and a previous occurrence metric, respectively.
